(19) United States
(12) Patent Application Publication
(10) Pub. No.: US 2004/0137076 A1
(43) Pub. Date: Jul. 15, 2004

(54) Antibacterial processing of fiber products

(76) Inventors: Naruki Yamauchi, Kyoto (JP); Katsuo Sasa, Kyoto (JP)

Correspondence Address: WENDEROTH, LIND & PONACK, L.L.P., 2033 K STREET N. W., SUITE 800, WASHINGTON, DC, 20006-1021, US

(21) Appl. No.: 10/742,768

(22) Filed: Dec. 23, 2003

(30) Foreign Application Priority Data
Apr. 8, 2002 (JP) 2002-104808

(51) Int. Cl. A61K 33/42; A61K 33/38

(52) U.S. Cl. 424/601; 424/618

(57) ABSTRACT

The invention provides an antibacterial processing agent for a fiber product comprising an inorganic antibacterial agent comprising an antibacterial metal carried on an inorganic carrier, The inorganic antibacterial agent is dispersed in water with an inorganic dispersing agent selected from the group consisting of boric acid, an alkali metal salt of boric acid and an alkali metal salt of phosphoric acid. The antibacterial processing agent is capable of providing a fiber product with antibacterial property with washing resistance without use of a binder resin.

### BACKGROUND OF THE INVENTION

1. Field of the Invention

The present invention relates to an antibacterial processing agent capable of providing a fiber product with antibacterial property with washing resistance without use of a binder resin. The antibacterial processing agent is a stable aqueous dispersion of a finely particulate inorganic antibacterial agent. The invention further relates to an antibacterial processing method using the antibacterial processing agent and an antibacterial fiber product obtained by using the antibacterial processing agent.

2. Description of Related Art

According to increasing interest in antibacterial processing in daily life, various fiber products are subjected to antibacterial processing and are put to practical use. The antibacterial processing for a fiber product has been carried out, for example, by a method of applying an antibacterial agent to a fiber product by post-processing, a method of mixing an antibacterial agent into fibers that form a fiber product, or a method of applying an antibacterial agent to a fiber product by using a binder resin. Examples of the antibacterial agent having been conventionally used include an organic antibacterial processing agent, such as a quaternary ammonium compound and a pyrithione compound, or a metal or a metal oxide having antibacterial activity, or an inorganic antibacterial processing agent formed by supporting them on a carrier, such as ceramics.

Among these antibacterial agents, the inorganic antibacterial agent is widely used particularly for antibacterial processing of fiber products. In the case where the inorganic antibacterial agent is applied in the form of an aqueous dispersion to a fiber product to provide the fiber product with antibacterial property, a large amount of a binder resin is necessarily used to obtain washing resistance, i.e., the antibacterial agent is retained in the processed fiber product to maintain the antibacterial activity upon washing the fiber product. The use of a binder resin brings about necessity of using a large amount of the antibacterial agent. Accordingly, the conventional antibacterial processing method using a binder resin has such a problem that the texture of the fiber product thus processed is deteriorated.

### SUMMARY OF THE INVENTION

The invention has been completed to solve the problems associated with the conventional antibacterial processing of a fiber product. It is therefore an object of the invention to provide an antibacterial processing agent in the form of a stable aqueous dispersion that is capable of providing a fiber product with antibacterial property with washing resistance without use of a binder resin. It is another object of the invention to provide an antibacterial processing method for a fiber product by using such an antibacterial processing agent as mentioned above, and an antibacterial fiber product obtained by using such an antibacterial processing agent.

The invention provides an antibacterial processing agent for a fiber product which comprises an inorganic antibacterial agent comprising an antibacterial metal carried on an inorganic carrier, the inorganic antibacterial agent being dispersed in water with an inorganic dispersing agent selected from the group consisting of boric acid, an alkali metal salt of boric acid and an alkali metal salt of phosphoric acid.

The invention also provides an antibacterial processing method for a fiber product comprising applying the antibacterial processing agent to a fiber product in the presence of at least one antibacterial processing auxiliary agent selected from the group consisting of a cationic surface active agent, a water soluble cationic resin and a cationic softening agent.

The invention further provides an antibacterial fiber product prepared by applying the antibacterial processing agent to a fiber product.

### DESCRIPTION OF THE PREFERRED EMBODIMENTS

The antibacterial processing agent for a fiber product according to the invention comprises an inorganic antibacterial agent comprising an antibacterial metal carried on an inorganic carrier, and the inorganic antibacterial agent is dispersed in water with an inorganic dispersing agent selected from the group consisting of boric acid, an alkali metal salt of boric acid and an alkali metal salt of phosphoric acid. The fiber product referred herein means fibers and textiles.

The inorganic antibacterial agent comprises an antibacterial metal carried on an inorganic carrier. Examples of the inorganic carrier include zeolite, zirconium phosphate, silica gel, apatite, hydrated titanium oxide, montmorillonite, glass powder and potassium titanate whiskers, but it is not limited to them.

Examples of the antibacterial metal include silver, copper, zinc, mercury, lead, bismuth, cadmium, chromium, cobalt, nickel and combinations of two or more of these metals, and preferred examples thereof include silver, zinc, copper and combinations of two or more of these metals. In particular, silver or a combination of silver and zinc is preferably used owing to the excellent antibacterial activity thereof.

Accordingly, preferred examples of the inorganic antibacterial agent used in the invention include zeolite having the aforementioned antibacterial metal carried thereon through ionic bond, zirconium phosphate having silver carried thereon through ion exchange, silica gel having silver carried thereon, apatite having silver carried thereon through ion exchange, hydrated titanium oxide having silver carried thereon, montmorillonite having silver carried thereon, glass powder having silver carried thereon, and potassium titanate whiskers having silver carried thereon, but it is not limited to them.

Further according to the invention, an inorganic antibacterial agent described, for example, in JP-A-6-239713 may also be used, which is obtained by depositing aluminum hydroxide on zeolite having an antibacterial metal carried thereon in an aqueous dispersion thereof, followed by heating.

The antibacterial processing agent of the invention is obtained by dispersing such an inorganic antibacterial agent as described above in water with an inorganic dispersing agent. As the inorganic dispersing agent, at least one selected from the group consisting of boric acid, an alkali metal salt of boric acid and an alkali metal salt of phosphoric acid is particularly used because of inactivity thereof to the antibacterial metal contained in the inorganic antibacterial agent. Specific examples thereof include boric acid, sodium metaborate, potassium borate, sodium phosphate, potassium phosphate, sodium hexametaphosphate, potassium hexametaphosphate, sodium tripolyphosphate, potassium tripolyphosphate, sodium pyrophosphate and potassium pyrophosphate. Among these, sodium hexametaphosphate or sodium metaborate is preferably used in the invention.

The inorganic dispersing agent may be used solely or in combination of two or more of them, and is generally used in an amount of from 0.03 to 10% by weight, and preferably from 0.1 to 5% by weight, based on the amount of the inorganic antibacterial agent.

The inorganic antibacterial agent can be generally dispersed in water in such a manner that the inorganic antibacterial agent is pulverized in the presence of the dispersing agent by using a wet pulverizer to an average particle diameter of the inorganic antibacterial agent of from 0.01 to 5 μm, and preferably from 0.01 to 1 μm, whereby the inorganic antibacterial agent can be stably dispersed in water. As a result, the antibacterial processing agent of the invention can be obtained in the form of a stable aqueous dispersion.

According to the invention, it is preferred from the practical standpoint that the antibacterial processing agent thus obtained contains the inorganic antibacterial agent in an amount of from 1 to 70% by weight, and particularly preferably from 1 to 40% by weight.

The antibacterial processing agent of the invention is a stable aqueous dispersion of the finely particulate inorganic antibacterial agent dispersed in water with the inorganic dispersing agent. According to the invention, a fiber product is provided with antibacterial activity with washing resistance without use of a binder resin by applying and adsorbing the antibacterial processing agent to a fiber product in the presence of at least one antibacterial processing auxiliary agent selected from the group consisting of a cationic surface active agent, a water soluble cationic resin and a cationic softening agent.

The cationic surface active agent used is selected from an amine salt, an ammonium salt, a phosphonium salt and a sulfonium salt. Specific examples of the amine salt or ammonium salt cationic surface active agent include an alkylimidazoline quaternary compound, a long-chain alkylpyridinium halide, an alkyltrimethylammonium halide, an alkoxymethylpyridinium halide, an alkyldimethylbenzylammonium halide, a polyoxyethylene alkylamine, an alkylamine acetate and a dialkyldimethylammonium halide. These may be used solely or in combination of two or more of them. Examples of the phosphonium salt cationic surface active agent include an alkylphosphonium halide, and examples of the sulfonium salt include an alkylsulfonium halide. In the invention, the amine salt and the ammonium salt are preferably used.

Examples of the water soluble cationic resin include cationized cellulose, such as hydroxycellulose-2-hydroxylpropyltrimethylammonium chloride ether.

A cationic softening agent is also preferably used as the antibacterial processing auxiliary agent. As having been well known, the softening agent is an additive used for imparting softness to a fiber product, and those having a cationic nature in ionicity among them are used in the invention. Examples of the cationic softening agent include an organic acid salt of a polyamine fatty acid amide compound, an amino-modified silicone polyamine fatty acid amide compound, a fatty acid condensate of a polyamine fatty acid amide compound quaternary salt and an amino-modified silicone, and also include emulsions obtained by emulsifying them with a cationic surface active agent or a nonionic surface active agent. These may be used solely or in combination of two or more of them.

Some of the aforementioned cationic surface active agents, for example, many of the quaternary ammonium salts, e.g., a dialkyldimethylammonium halide, can be used as the cationic softening agent by themselves.

According to the invention, the antibacterial processing agent may be applied to a fiber product in the presence of the cationic softening agent, whereby softness and antistatic property can be applied to a fiber product simultaneously with antibacterial activity. The antibacterial processing auxiliary agent is used usually in an amount of from 0.001 to 10.0% by weight, and preferably from 0.05 to 2.0% by weight, based on the amount of the inorganic antibacterial agent.

According to the invention, the applied amount of the inorganic antibacterial agent to a fiber product is usually from 0.01 to 1.0% by weight based on the fiber product. In the case where the applied amount of the inorganic antibacterial agent is less than 0.01% by weight based on the fiber product, satisfactory antibacterial function is not imparted to the fiber product. On the other hand, in the case where it is applied in an amount exceeding 1% by weight, it is not preferred since the antibacterial function of the fiber product is not improved corresponding to the increased amount, and since the cost for the antibacterial processing is increased.

The antibacterial processing method of the invention is one of the post-treating methods, and specifically, it is particularly preferably carried out, for example, by an adsorption method. Accordingly, as similar to the conventional dyeing method, the antibacterial processing agent and the antibacterial processing auxiliary agent are placed in a dyeing apparatus along with a fiber product, and the whole of the antibacterial agent is adsorbed on the fiber product by heating to a temperature of from 70 to 100° C. It is also possible that a dye is simultaneously placed therein, whereby the antibacterial processing is carried out under the same conditions as the dyeing conditions.

Because the antibacterial processing method of the invention is an adsorption method among the post-processing methods, a fiber product is imparted antibacterial property by using only a smaller amount of the inorganic antibacterial agent with respect to the fiber product without loss of the antibacterial agent, and furthermore, deterioration in texture due to the antibacterial processing can be suppressed, in comparison to such processing methods as the kneading method and a method using a binder resin in combination.

Upon preparation of the antibacterial processing agent of the invention, an organic dispersing agent may be used in combination with the aforementioned inorganic dispersing agent in such an amount that does not impair the antibacterial processing agent thus obtained. Furthermore, various kinds of additives, such as a water repellent agent and an antistatic agent, may be contained depending on necessity in the antibacterial processing agent thus obtained, and a softening agent other than the aforementioned cationic softening agent, such as a nonionic surface active agent, may also be contained.

### EXAMPLES

The invention will be described in detail with reference to the following examples and comparative examples, but the invention is not construed as being limited thereto. All percents and parts in the examples and the comparative examples are those by weight. The antibacterial products obtained in the examples and the comparative examples were evaluated for antibacterial activity by the following methods.

Average Particle Diameter of Inorganic Antibacterial Agent in Antibacterial Processing Agent

The particle size distribution of the inorganic antibacterial agent in the antibacterial processing agent was measured by using a laser diffraction particle size distribution measuring apparatus, SALD-2000J, manufactured by Shimadzu Corp., and a median diameter obtained was designated as the average particle diameter.

Antibacterial Activity

A cloth (sample) was subjected to an antibacterial processing and was then washed with water under the following conditions. The cloth was then subjected to measurement for antibacterial activity by the SEK Test Method. According to the SEK Test Method, the following requirements must be satisfied so that antibacterial processing carried out is effective.

Antibacterial deodorization standard:

bacteriostatic activity (log B/C)≧2.2

Bacterial control standard: C≦A (C≠0)

wherein A represents the number of bacteria recovered immediately after inoculation to the standard cloth (nylon), B represents the number of bacterial recovered after culture of the standard cloth (nylon) for 18 hours (it is defined that the test is approved when the condition (log B/A)>1.5 is satisfied), and C represents the number of bacteria recovered after culture of an antibacterial and deodorized cloth or a bacterial controlled cloth for 18 hours.

Washing with Water

According to JIS L0217 103, after a sample was washed with water using the JAFET Standard Detergent in an amount of 3 g/L with a bath ratio of 1/30 at 40° C. for 5 minutes, the sample was rinsed twice at 40° C. for 2 minutes, respectively, and subjecting the sample to centrifugal dehydration, followed by drying in shade. The aforementioned operation was carried out 10 times.

### EXAMPLE 1

An inorganic antibacterial agent obtained by ionically bonding an antibacterial metal to zeolite was pulverized to fine particles in a wet pulverizer by using 5% of sodium hexaphosphate based on the weight of the inorganic antibacterial agent as an inorganic dispersing agent, and was dispersed in water to obtain an aqueous dispersion containing 20% of the inorganic antibacterial agent having an average particle diameter of 0.3 μm, which was designated as an antibacterial processing agent A.

### EXAMPLE 2

An inorganic antibacterial agent obtained by ionically bonding an antibacterial metal to zeolite was pulverized to fine particles in a wet pulverizer by using 5% of sodium metaborate based on the weight of the inorganic antibacterial agent as an inorganic dispersing agent, and was dispersed in water to obtain an aqueous dispersion containing 20% of the inorganic antibacterial agent having an average particle diameter of 0.4 μm, which was designated as an antibacterial processing agent B.

### EXAMPLE 3

An inorganic antibacterial agent obtained by carrying silver on potassium titanate whiskers was pulverized to fine particles in a wet pulverizer by using 5% of sodium hexaphosphate based on the weight of the inorganic antibacterial agent as an inorganic dispersing agent, and was dispersed in water to obtain an aqueous dispersion containing 20% of the inorganic antibacterial agent having an average particle diameter of 0.3 μm, which was designated as an antibacterial processing agent C.

### COMPARATIVE EXAMPLE 1

An inorganic antibacterial agent obtained by ionically bonding an antibacterial metal to zeolite was pulverized to fine particles in a wet pulverizer by using 5% of a polyoxyethylene phenyl ether nonionic surface active agent based on the weight of the inorganic antibacterial agent as a dispersing agent, and was dispersed in water to obtain an aqueous dispersion containing 20% of the inorganic antibacterial agent having an average particle diameter of 0.5 μm, which was designated as an antibacterial processing agent D.

### COMPARATIVE EXAMPLE 2

An inorganic antibacterial agent obtained by ionically bonding an antibacterial metal to zeolite was pulverized to fine particles in a wet pulverizer by using 5% of a dialkyl sulfosuccinate anionic surface active agent based on the weight of the inorganic antibacterial agent as a dispersing agent, and was dispersed in water to obtain an aqueous dispersion containing 20% of the inorganic antibacterial agent having an average particle diameter of 0.6 μm, which was designated as an antibacterial processing agent E.

### EXAMPLE 4

A polyester fiber net was subjected to antibacterial processing as a sample by using 2% owf (on the weight of fabric) of the antibacterial agent A produced in Example 1 and 0.03% owf of alkyldimethylbenzylammonium chloride as an antibacterial processing auxiliary agent. Specifically, at a bath ratio of 1/30, the temperature was increased from 40° C. to 70° C. at a rate of 3° C. per minute and maintained at that temperature for 20 minutes to attain underwater treatment, and thereafter, the temperature of the treating bath was lowered to 40° C. The thus processed sample was then washed, dehydrated and dried to obtain an antibacterial treated product.

### EXAMPLE 5

The antibacterial processing was carried out in the same manner as in Example 4 except that nylon stockings were used instead of the polyester fiber net.

### EXAMPLE 6

A polyester fiber net was subjected to antibacterial processing in the same manner as in Example 4 except that the antibacterial processing agent B produced in Example 2 was used instead of the antibacterial processing agent A.

### EXAMPLE 7

A polyester fiber net was subjected to antibacterial processing in the same manner as in Example 4 except that the antibacterial processing agent C produced in Example 3 was used instead of the antibacterial processing agent A.

### COMPARATIVE EXAMPLE 3

A polyester fiber net was subjected to antibacterial processing in the same manner as in Example 4 using the antibacterial processing agent A but using no alkyldimethylbenzylammonium chloride as an antibacterial processing auxiliary agent.

### COMPARATIVE EXAMPLE 4

A polyester fiber net was subjected to antibacterial processing in the same manner as in Example 4 except that the antibacterial processing agent D produced in Comparative Example 1 was used instead of the antibacterial processing agent A, and nylon stockings were used as a sample instead of the polyester fiber net.

### COMPARATIVE EXAMPLE 5

A polyester fiber net was subjected to antibacterial processing in the same manner as in Example 4 except that the antibacterial processing agent E produced in Comparative Example 2 was used instead of the antibacterial processing agent A, and nylon stockings were used as a sample instead of the polyester fiber net.

The samples thus subjected to antibacterial processing in Examples and Comparative Examples in the initial state (L=0) and after 10 times washing (L=10) were evaluated for antibacterial activity to Staphylococcus aureus and Klebsiella pneumoniae according to the SEK Test Method. The results of evaluation for antibacterial activity to Staphylococcus aureus are shown in Table 1, and the results of evaluation for antibacterial activity to Klebsiella pneumoniae are shown in Table 2.

TABLE 1

| | | | Viable cell count | | | | Increasing and | |
|---|---|---|---|---|---|---|---|---|
| | | | Immediately after | 18 hours after | Viable cell count | | decreasing count | |
| | | | inoculation (A) | inoculation (B) | log A | log B | (log B/A) | |
| Standard cloth (nylon) | | | 2.2 × 10⁴ | 1.6 × 10⁷ | 4.3 | 7.2 | 2.9 | |
| | | | | 18 hours after | | | Bacteriostatic | Sterilized |
| | | | | inoculation (C) | | log C | activity | activity |
| Example 4 | Treated sample | L = 0 | | 20 or less | | 1.3 or more | 5.9 or more | 3.0 or more |
| | | L = 10 | | 20 or less | | 1.3 or more | 5.9 or more | 3.0 or more |
| Example 5 | Treated sample | L = 0 | | 20 or less | | 1.3 or more | 5.9 or more | 3.0 or more |
| | | L = 10 | | 20 or less | | 1.3 or more | 5.9 or more | 3.0 or more |
| Example 6 | Treated sample | L = 0 | | 20 or less | | 1.3 or more | 5.9 or more | 3.0 or more |
| | | L = 10 | | 20 or less | | 1.3 or more | 5.9 or more | 3.0 or more |
| Example 7 | Treated sample | L = 0 | | 20 or less | | 1.3 or more | 5.9 or more | 3.0 or more |
| | | L = 10 | | 20 or less | | 1.3 or more | 5.9 or more | 3.0 or more |
| Comparative | Treated sample | L = 0 | | 1.2 × 10⁵ | | 5.0 | 2.2 | −0.7 |
| Example 3 | | L = 10 | | 9.0 × 10⁷ | | 7.9 | −0.3 | −3.6 |
| Comparative | Treated sample | L = 0 | | 9.6 × 10⁴ | | 4.9 | 2.3 | −0.6 |
| Example 4 | | L = 10 | | 3.2 × 10⁷ | | 7.5 | −0.3 | −3.2 |
| Comparative | Treated sample | L = 0 | | 3.2 × 10⁷ | | 4.7 | 2.5 | −0.4 |
| Example 5 | | L = 10 | | 2.9 × 10⁶ | | 6.4 | 0.8 | −2.1 |

TABLE 2

| | | | Viable cell count | | | | Increasing and | |
|---|---|---|---|---|---|---|---|---|
| | | | Immediately after | 18 hours after | Viable cell count | | decreasing count | |
| | | | inoculation (A) | inoculation (B) | log A | log B | (log B/A) | |
| Standard cloth (nylon) | | | 2.2 × 10⁴ | 1.6 × 10⁷ | 4.3 | 7.2 | 2.9 | |
| | | | | 18 hours after | | | Bacteriostatic | Sterilized |
| | | | | inoculation (C) | | log C | activity | activity |
| Example 4 | Processed sample | L = 0 | | 20 or less | | 1.3 or more | 5.9 or more | 3.0 or more |
| | | L = 10 | | 20 or less | | 1.3 or more | 5.9 or more | 3.0 or more |
| Example 5 | Processed sample | L = 0 | | 20 or less | | 1.3 or more | 5.9 or more | 3.0 or more |
| | | L = 10 | | 20 or less | | 1.3 or more | 5.9 or more | 3.0 or more |
| Example 6 | Processed sample | L = 0 | | 20 or less | | 1.3 or more | 5.9 or more | 3.0 or more |
| | | L = 10 | | 20 or less | | 1.3 or more | 5.9 or more | 3.0 or more |
| Example 7 | Processed sample | L = 0 | | 20 or less | | 1.3 or more | 5.9 or more | 3.0 or more |
| | | L = 10 | | 20 or less | | 1.3 or more | 5.9 or more | 3.0 or more |
| Comparative | Processed sample | L = 0 | | 1.2 × 10⁵ | | 5.0 | 2.2 | −0.7 |
| Example 3 | | L = 10 | | 9.0 × 10⁷ | | 7.9 | −0.3 | −3.6 |
| Comparative | Processed sample | L = 0 | | 1.1 × 10⁵ | | 5.0 | 2.2 | −0.7 |
| Example 4 | | L = 10 | | 9.0 × 10⁶ | | 7.0 | 0.5 | −2.6 |
| Comparative | Processed sample | L = 0 | | 8.4 × 10⁴ | | 4.9 | 2.3 | −0.6 |
| Example 5 | | L = 10 | | 3.2 × 10⁶ | | 6.5 | 1.0 | −2.1 |

### EXAMPLES 8-10 AND COMPARATIVE EXAMPLES 6-12

In Examples 8 to 9 and Comparative Examples 6 to 11, zeolite having an antibacterial metal ionically bonded thereto was used as the inorganic antibacterial agent, and in Example 10 and Comparative Example 12, potassium titanate whiskers having silver carried thereon are used as the inorganic antibacterial agent.

The inorganic antibacterial agent was dispersed in water by using a dispersing agent or by using no dispersing agent as shown in Tables 3 to 5 to prepare an antibacterial processing agent. As a sample, nylon fiber stockings were subjected to antibacterial processing by using the antibacterial processing agent in an amount of 3% owf and using an antibacterial processing auxiliary agent in an amount of 0.03% owf or using no antibacterial processing auxiliary agent as shown in Tables 3 to 5. Specifically, at a bath ratio of 1/20, the temperature was increased from 40° C. to 70° C. at a rate of 1° C. per minute and maintained at that temperature for 10 minutes to attain underwater treatment, and thereafter, the temperature of the treating bath was lowered to 40° C. The thus processed sample was then washed, dehydrated and dried to provide an antibacterial fiber product.

The antibacterial processing auxiliary agent a used herein was alkyldimethylbenzylammonium chloride (cationic surface active agent), b was hydroxycellulose-2-hydroxypropyltrimethylammonium chloride ether (water soluble cationic resin), c was an organic acid salt of a polyamine fatty acid amide compound (cationic softening agent), d was sulfosuccinic acid dialkyl salt (anionic surface active agent), and e was carboxymethyl cellulose sodium salt (anionic surface active agent).

The adsorption property of the antibacterial agent to the antibacterial treated products thus obtained and the washing resistance of the antibacterial treated products are shown in Tables 3 to 5. The adsorption property of the antibacterial agent to the antibacterial treated products and the washing resistance of the antibacterial treated products were evaluated in the following manner. The antibacterial processing agent, which is an aqueous dispersion of the antibacterial agent dispersed in water, is white clouded before subjecting antibacterial processing of a sample, and after completing the antibacterial processing, the treating agent becomes transparent if the antibacterial agent is adsorbed on the sample, but the treating agent remains white clouded if the antibacterial agent is not adsorbed on the sample. Accordingly, it was decided that adsorption of the antibacterial agent to a sample was favorably attained in the case where the treating agent after completing the antibacterial processing was transparent (shown by symbol A), adsorption of the antibacterial agent to a sample was insufficient in the case where the treating agent remained slightly white clouded (shown by symbol B), and substantially no adsorption of the antibacterial agent to a sample was attained in the case where the treating agent was white clouded as similar to that before the treatment (shown by symbol C).

The washing resistance of antibacterial samples thus prepared was evaluated in the following manner. The antibacterial sample was immersed in washing water (hot water at 80° C.) for 30 minutes, and then stood to cool to room temperature. The washing water remained transparent in the case where the antibacterial agent was still adsorbed on the sample, which meant good washing resistance (shown by symbol A), and the washing water became white clouded in the case where the antibacterial agent was released from the sample, which meant poor washing resistance (shown by symbol C).

TABLE 3

| | | Average | | Antibacterial activity | |
|---|---|---|---|---|---|
| | | particle | | of antibacterial | |
| | | diameter of | | product | |
| | | antibacterial | Auxiliary | Adsorption | Washing |
| | Dispersing agent | agent (μm) | agent | property | resistance |
| Example 8 | sodium | 0.3 | a | A | A |
| | hexametaphosphate | | b | A | A |
| | | | c | A | A |
| Comparative | sodium | 0.3 | d | A (*) | C |
| Example 6 | hexametaphosphate | | e | C | — |
| | | | none | C - B | C |
| Comparative | nonionic surface | 0.5 | a | C - B | C |
| Example 7 | active agent | | b | C | — |
| | (polyoxyethylene | | c | C | — |
| | alkyl ether) | | d | C | — |
| | | | e | C | — |
| | | | none | B - C | C |

Note:
(*) The antibacterial agent in the antibacterial treating agent was aggregated.

TABLE 4

| | | Average | | Antibacterial activity | |
|---|---|---|---|---|---|
| | | particle | | of antibacterial | |
| | | diameter of | | product | |
| | | antibacterial | Auxiliary | Adsorption | Washing |
| | Dispersing agent | agent (μm) | agent | property | resistance |
| Comparative | nonionic surface | 0.3 | a | C - B | C |
| Example 8 | active agent | | b | A - B | C |
| | (dialkyl | | c | C | — |
| | sulfosuccinate | | d | C | — |
| | type) | | e | C | — |
| | | | none | C | — |
| Comparative | cationic surface | 0.5 | a | C | — |
| Example 9 | active agent | | b | C | — |
| | (special carboxylic | | c | C | — |
| | acid polymer | | d | C | — |
| | surface active | | e | C | — |
| | agent) | | none | C | — |
| Comparative | none | 0.8 | a | C | — |
| Example 10 | | | b | A⁽*⁾ | — |
| | | | c | C | — |
| | | | d | C | — |
| | | | e | C | — |
| | | | none | C | — |

Note:
⁽*⁾The antibacterial agent in the antibacterial treating agent was aggregated.

TABLE 5

| | | Average | | Antibacterial activity | |
|---|---|---|---|---|---|
| | | particle | | of antibacterial | |
| | | diameter of | | product | |
| | | antibacterial | Auxiliary | Adsorption | Washing |
| | Dispersing agent | agent (μm) | agent | property | resistance |
| Example 9 | sodium | 0.4 | a | A | A |
| | metaphosphate | | b | A | A |
| | | | c | A | A |
| Comparative | sodium | 0.4 | d | C | — |
| Example 11 | metaphosphate | | e | C | — |
| | | | none | C - B | C |
| Example 10 | sodium | 0.3 | a | A | A |
| | hexametaphosphate | | b | A | A |
| | | | c | A | A |
| Comparative | sodium | 0.3 | d | C | — |
| Example 12 | hexametaphosphate | | e | C | — |
| | | | none | B | — |

Note:
(*) The antibacterial agent in the antibacterial treating agent was aggregated.

In the case where a fiber product is subjected to antibacterial processing by using the antibacterial processing agent according to the invention and using at least one antibacterial processing auxiliary agent selected from the group consisting of a cationic surface active agent, a water soluble cationic resin and a cationic softening agent, the antibacterial agent can be firmly adsorbed on the fiber product, and therefore, the antibacterial agent is prevented from being released from the fiber product having been subjected to the antibacterial processing even when the fiber product is washed. Consequently, the resulting antibacterial treated fiber product has excellent washing resistance with respect to the antibacterial processing thus attained.

In the case where an anionic surface active agent is used as the antibacterial processing auxiliary agent, on the other hand, the antibacterial agent is insufficiently adsorbed on a fiber product, and even though the antibacterial agent is adsorbed on a fiber product, it is easily released from the fiber product upon washing to exhibit deteriorated washing resistance with respect to the antibacterial processing thus attained. In the case where no antibacterial processing auxiliary agent is used in the antibacterial processing, no antibacterial agent can be generally adsorbed on a fiber product.

As described in the foregoing, the antibacterial processing agent according to the invention is formed with an aqueous dispersion of a finely particulate inorganic antibacterial agent without a binder resin and can easily apply antibacterial processing to a fiber product by an adsorption treating method, and furthermore, an antibacterial treated fiber product thus obtained has high antibacterial activity and is excellent in washing resistance.

What is claimed is:

1. An antibacterial processing agent for a fiber product comprising an inorganic antibacterial agent comprising an antibacterial metal carried on an inorganic carrier, the inorganic antibacterial agent being dispersed in water with an inorganic dispersing agent selected from the group consisting of boric acid, an alkali metal salt of boric acid and an alkali metal salt of phosphoric acid.

2. An antibacterial processing agent as claimed in claim 1, wherein the inorganic dispersing agent is at least one selected from the group consisting of boric acid, sodium metaborate, potassium borate, sodium phosphate, potassium phosphate, sodium hexametaphosphate, potassium hexametaphosphate, sodium tripolyphosphate, potassium tripolyphosphate, sodium pyrophosphate and potassium pyrophosphate.

3. An antibacterial processing agent as claimed in claim 1, wherein the inorganic antibacterial agent is at least one selected from the group consisting of zeolite having an antibacterial metal carried thereon, zirconium phosphate having silver carried thereon, silica gel having silver carried thereon, apatite having silver carried thereon, hydrated titanium oxide having silver carried thereon, montmorillonite having silver carried thereon, glass powder having silver carried thereon, and potassium titanate whiskers having silver carried thereon.

4. An antibacterial processing agent as claimed in claim 3, wherein the inorganic antibacterial agent has an average particle diameter of from 0.01 to 5 μm.

5. An antibacterial processing agent as claimed in claim 1, which comprises the inorganic dispersing agent in an amount of from 0.03 to 10% by weight and the inorganic antibacterial agent in an amount of from 1 to 70% by weight.

6. An antibacterial processing method comprising applying an antibacterial processing agent as claimed in claim 1 to a fiber product in the presence of at least one antibacterial processing auxiliary agent selected from the group consisting of a cationic surface active agent, a water soluble cationic resin and a cationic softening agent.

7. An antibacterial processing method as claimed in claim 6, wherein the cationic surface active agent is at least one selected from the group consisting of an amine salt, an ammonium salt, a phosphonium salt and a sulfonium salt.

8. An antibacterial processing method as claimed in claim 6, wherein the cationic surface active agent is at least one selected from the group consisting of an alkylimidazoline quaternary compound, a long-chain alkylpyridinium halide, an alkyltrimethylammonium halide, an alkoxymethylpyridinium halide, an alkyldimethylbenzylammonium halide, a polyoxyethylene alkylamine, an alkylamine acetate and a dialkyldimethylammonium halide.

9. An antibacterial processing method as claimed in claim 6, wherein the cationic softening agent is at least one selected from the group consisting of an organic acid salt of a polyamine fatty acid amide compound, an amino-modified silicone polyamine fatty acid amide compound, a fatty acid condensate of a polyamine fatty acid amide compound quaternary salt and an amino-modified silicone, and emulsions obtained by emulsifying them with a cationic surface active agent or a nonionic surface active agent.

10. An antibacterial processing method as claimed in claim 6, wherein the water soluble cationic resin is cationized cellulose.

11. An antibacterial processing method as claimed in claim 6, wherein the antibacterial processing auxiliary agent is used in an amount of from 0.001 to 10.0% by weight based on the inorganic antibacterial agent.

12. An antibacterial treated fiber product, to which an antibacterial processing agent as claimed in claim 1 is applied.

13. An antibacterial treated fiber product having been subjected to antibacterial processing by a method as claimed in claim 6
